(19) United States
(12) Patent Application Publication
(10) Pub. No.: US 2016/0022206 A1
(43) Pub. Date: Jan. 28, 2016

(54) MULTI-MODAL PHARMACO-DIAGNOSTIC ASSESSMENT OF BRAIN HEALTH

(71) Applicants: Adam J. SIMON, Yardley, PA (US); Stephen J. MARTINO, Farmingdale, NJ (US)

(72) Inventors: Adam J. SIMON, Yardley, PA (US); Stephen J. MARTINO, Farmingdale, NJ (US)

(21) Appl. No.: 14/773,872

(22) PCT Filed: Mar. 14, 2014
(86) PCT No.: PCT/US14/26962

§ 371 Date: Sep. 9, 2015

Related U.S. Application Data
(60) Provisional application No. 61/794,143, filed on Mar. 15, 2013.
(60) Provisional application No. 61/864,864, filed on Aug. 12, 2013.

(51) Int. Cl. A61B 5/00; A61B 19/00

(52) U.S. Cl. CPC A61B 5/4848; A61B 5/4064; A61B 5/4824; A61B 5/4094; A61B 5/4082; A61B 5/4088; A61B 19/5225; A61B 2503/10; A61B 2503/22

(57) ABSTRACT

A single diagnostic dose of a chemical agent that can bind with molecular specificity or provide a well characterized molecular effect on a mammalian host (including humans) is provided to a patient between brain scans. The method typically comprises at least one pre-dose scan of the subject followed by a waiting period then a second post-dose diagnostic scan. The diagnostic scans can be conventional in nature or of a multi-modal variety. A comparison, in the form of a difference or ratio, between data or extracted features before versus after the diagnostic dose indicates with molecular specificity the tone in the brain of that subject. The resulting data may be used to assess instances of medical fraud and can be used in back to work decisions for brain and soft tissue injuries for which the determinations have traditionally been somewhat subjective in nature.

### CROSS REFERENCE TO RELATED APPLICATIONS

This application claims benefit of U.S. Provisional Application No. 61/794,143 filed Mar. 15, 2013, and of U.S. Provisional Application No. 61/864,864 filed Aug. 12, 2013. The contents of these patent applications are hereby incorporated by reference in their entireties.

### TECHNICAL FIELD

The invention relates to the diagnosis and analysis of brain health information through the use of activated tasks and stimuli in combination with single doses of approved drugs, biologics, and biochemical CNS active agents in a system to dynamically assess brain health, function and cerebral activity.

### BACKGROUND

Normal functioning of the brain and central nervous system is critical to a healthy, enjoyable and productive life. Disorders of the brain and central nervous system are among the most dreaded of diseases. Many neurological disorders such as Alzheimer's disease and Parkinson's disease are insidious and progressive, becoming more common with increasing age. While such a disorder like a stroke occurs abruptly, it is also more common with increasing age. Others such as schizophrenia, depression, Autism, multiple sclerosis and epilepsy arise at younger age and can persist and progress throughout an individual's lifetime. Sudden catastrophic damage to the nervous system, such as brain trauma, infections and intoxications can also affect any individual of any age at any time.

Most nervous system dysfunction arises from complex interactions between an individual's genotype, environment and personal habits and can manifest in a personalized presentation. However, despite the emerging importance of preventative health care, convenient means for objectively assessing the health of one's own central nervous system have not been widely available. Therefore, new ways to monitor the health status of the brain and central nervous system are needed for routine health surveillance, early diagnosis of dysfunction, tracking of disease progression and the discovery and optimization of treatments and new therapies.

Unlike cardiovascular and metabolic disorders, where personalized health monitoring biomarkers such as blood pressure, cholesterol, and blood glucose have long become household terms, no such convenient biomarkers of the brain and central nervous system health exist. Quantitative neurophysiological assessment techniques and approaches such as positron emission tomography (PET), functional magnetic resonance imaging (fMRI) and neuropsychiatric or cognition testing involve significant operator expertise, inpatient or office-based testing, significant time and significant expense. One potential technique that may be adapted to serve a broader role as a facile biomarker of the central nervous system function is a multi-modal assessment of the brain from a number of different forms of data, including electroencephalography (EEG), which measures the brain's electrical activity. However, formal clinical lab-based EEG approaches typically require significant operator training, cumbersome and expensive equipment, and a dedicated area for testing and are used primarily to evaluate patients for seizures, cognitive slowing and brain death.

Alternate and innovative biomarker approaches are needed to provide quantitative measurements of personal brain health that could greatly improve the prevention, diagnosis and treatment of neurological and psychiatric disorders. Unique multi-modal devices and tests that lead to biomarkers of Parkinson's disease, Alzheimer's disease, concussion and other central nervous system pathology and neuropsychiatric conditions is a pressing need.

### SUMMARY

The systems and methods of the invention utilize a single diagnostic dose of a chemical agent such as a regulatory agency approved drug, biologic, vitamin, ingredient, or other chemical or biologic agent that can bind with molecular specificity or provide a well characterized molecular effect on a mammalian host (including humans). The method typically involves at least one pre-dose multi-modal diagnostic scan of the subject followed by a waiting period, on the order of minutes to a couple of hours, after the point in time when the chemical agent is taken. Thereafter, a second post-dose multi-modal diagnostic scan will take place. A comparison, in the form of a difference or ratio, between data streams or extracted features from the data streams before versus after the diagnostic dose of the chemical agent will indicate with molecular specificity the tone in the brain of that subject to the molecular agent. In another embodiment, the multi-modal diagnostic scan could be replaced by more traditional neuro-diagnostic techniques such as EEG, MRI, PET, CT, SPECT, MEG, fMRI, MRS and other neuro-diagnostic modalities in order to provide molecular specificity to those traditional neuro-diagnostics modalities.

The systems and methods of the invention enable one to use one or more than one molecular agent to assess the brain's response across multiple neurotransmitter systems. Thus, one could choose to probe the cholinergic, dopaminergic, serotonin, norepinephrine, glutaminergic and GABA systems. This can be accomplished either in a single multi-agent cocktail of single dose agents (complicated by possible drug-drug interactions) or sequential single dose assessments conducted after sufficient wash-out of each agent to insure that each compound was probing uniquely for evidence of neurotransmitter system deficits or disorder.

In specific embodiments, selective serotonin reuptake inhibitors (SSRI) as a class of pharmaco-diagnostic agents could be used to probe the serotonergic tone of a human subject's brain as part of a multi-modal diagnostic response (or traditional neuro-diagnostics response) to a single dose of one of these agents, especially in the case of someone suspected of a concussion and/or traumatic brain injury. The systems and methods may also be used with approved therapeutic agents for Alzheimer's disease, including the class of acetylcholinesterase inhibitors. In addition, an NMDA-receptor antagonist like Memantine could alternatively be used to probe the tone in an alternate neurotransmitter system for important complementary or standalone diagnostic information. The systems and methods of the invention may also be used with epilepsy and seizure management agents to aid in the diagnosis of seizures or epilepsy, identify those at risk for seizures or epilepsy, and evaluate a patient for potential efficacy of an epilepsy and seizure management agent. NDMA and GABA therapeutic drugs in single diagnostic dose form also may be used to probe the tone of the developing human brain, in particular for evidence of autism spectrum disorders in infants and toddlers.

In yet another embodiment of the invention, regulatory agency approved SSRI therapeutic class agents may be used to aid in probing the serotonin tone, in particular for neuropsychiatric conditions like depression. Approved Gamma-Aminobutyric acid (GABA) “GABAinergic” agents may also be used to aid in probing the GABAinergic tone, in particular for neuropsychiatric conditions like anxiety, schizophrenia, bi-polar disorder as well as pain and the cerebral cortex. Antipsychotic agents may also be used to probe the brain tone, in particular for neuropsychiatric conditions like anxiety, schizophrenia, schizoaffective disorders, OCD, Tourette's disease, tic disorders, bi-polar disorder and other mental health issues dealing with delusions, hallucinations, or disordered or disorganized thoughts. Approved stimulant agents, such as methylphenidate or dextro-amphetamine, may also be used to aid in probing the tone of the brain. Non-stimulants also could offer interesting diagnostic information from agents such as atomoxetine that could be used to probe the tone of a human subject's brain to test the multi-modal diagnostic response to a single dose of one of these agents. The systems and methods of the invention may also be used with approved norepinephrine agents to aid in probing the Norepinephrinergic tone, in particular for neuropsychiatric conditions like depression, anxiety, schizophrenia, bi-polar disorder, ADHD and narcolepsy.

Another embodiment of the present invention includes the use of a multi-modal brain assessment system (or a more traditional modality) at (i) a physician's offices, (ii) field-derived locations or (iii) at home in order to proctor these brain health scans on a regular, longitudinal basis, either with or without use of a pharmaco-diagnostic agent, in order to monitor a Sickle Cell disease patient's brain for evidence of reduced or abnormal activity. Such a shift would provide diagnostic information to indicate a subject should get a blood transfusion. The present invention also includes the use of multi-modal neuro diagnostic scanning, either with or without a pharmaco-diagnostic CNS active agents, to provide prognostic biomarkers of, for example, a post anoxic encephalopathy subject who is in the emergency department or intensive care unit of a hospital, physician office or clinic. Prognostic markers could enable clinicians to predict patient outcomes and enable appropriate clinical decisions in light of the prognostic information. In particular, cardiologists, intensivists and neurologists may find the present invention useful in any patient who presents after an anoxic cerebral event such as, but not limited to, cardiac arrest, cardiac arrhythmia, near-drowning, respiratory failure or suicide attempt.

In still other embodiments of the present invention, multi-modal neuro-diagnostic scanning, either with or without pharmaco-diagnostic CNS active agents, is used to provide objective clinical evidence of impaired brain health or a lack thereof in relation to workers compensation insurance cases or Medicare insurance benefits related to brain injury and disorders. Because certain brain injuries are “invisible” as defined by the patient manifesting only subjective complaints without objective, corroborating diagnostic evidence, it is very difficult today to tell who is legitimately hurt and in need of appropriate medical care and who is malingering and should get off insurance and return to work. Objective multi-modal diagnostic biomarker evidence from the present invention could enable clinicians and thus insurance companies to understand with objective clinical evidence who has suffered a brain-related injury and/or is still suffering from this injury. Such multi-modal neuro-diagnostic scanning, either with or without pharmaco-diagnostic CNS active agents, also may be used to provide objective clinical evidence of fatigue and lethargy. Memantine or another NMDA receptor antagonist also may be used to treat patients diagnosed with a concussion or mild Traumatic Brain Injury.

In yet other embodiments, multi-modal neuro-diagnostic scanning is used with dopaminergic agents to create a time series of the effectiveness of such agents to a subject to, for example, determine if that subject should enroll in a clinical trial of that agent. Such techniques may also be used to screen different medications for effectiveness for a given subject and to provide data for use in predictive analytics exercises for other subjects considering use of the same medications. This latter technique may be used to cut short therapy selections for new patients.

### BRIEF DESCRIPTION OF THE DRAWINGS

Embodiments of the invention can be better understood with reference to the following figures:

FIG. 1 is a schematic diagram illustrating in the upper timeline the pre and post diagnostic dose temporal scan sequence to practice the present invention. The lower timeline illustrates how the pre-post diagnostic dose scan sequence can be used on an every other day basis from home without the need to visit a hospital, clinic or doctor's office.

FIG. 2 is a schematic diagram illustrating how the present invention could be used to manage return to work, return to learn, or return to driving decisions, in a similar fashion to return to play decisions for athletes or return to duty decisions for soldiers. This could be generally called return-to-activity decisions.

FIG. 3 is a schematic diagram illustrating how the present invention could be used to manage risk and identify potential fraud in a work stream of claims within an insurance company for worker's compensation and other injury related claims.

### DETAILED DESCRIPTION OF ILLUSTRATIVE EMBODIMENTS

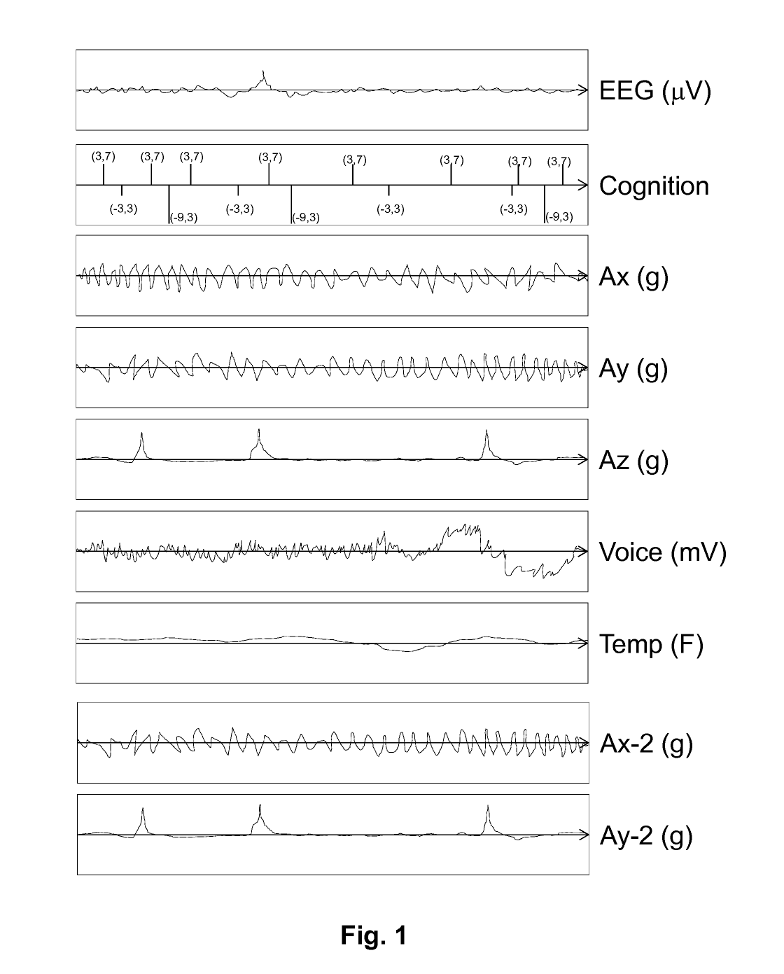

The invention will be described in detail below with reference to FIGS. 1-3. Those skilled in the art will appreciate that the description given herein with respect to FIGS. 1-3 is for exemplary purposes only and is not intended in any way to limit the scope of the invention. All questions regarding the scope of the invention may be resolved by referring to the appended claims.

### DEFINITIONS

By “electrode to the scalp” we mean to include, without limitation, those electrodes requiring gel, dry electrode sensors, contactless sensors and any other means of measuring the electrical potential or apparent electrical induced potential by electromagnetic means.

By “monitor the brain and central nervous system” we mean to include, without limitation, surveillance of normal brain health and aging, the early detection and monitoring of brain dysfunction, detection and monitoring of brain injury and recovery, identification of disease onset, progression and response to therapy, for the discovery and optimization of treatment and drug therapies, including without limitation, monitoring investigational compounds and registered pharmaceutical agents, as well as the monitoring of illegal substances and their presence or influence on an individual while driving, playing sports, or engaged in other regulated behaviors.

A “medical therapy” as used herein is intended to encompass any form of therapy with a potential biological, physiologic or biochemical effect, including, without limitation, any pharmaceutical agent or treatment, compounds, biologics, medical device therapy, exercise, biofeedback or combinations thereof.

By “EEG data” we mean to include without limitation the raw time series, any spectral properties determined after Fourier transformation, any nonlinear properties after nonlinear analysis, any wavelet properties, any summary biometric variables and any combinations thereof.

A “sensory and cognitive challenge” as used herein is intended to encompass any form of sensory stimuli (to the five senses), cognitive challenges (to the mind), and other physiological challenges (such as a respiratory CO₂ challenge, virtual reality balance challenge, hammer to knee reflex challenge, etc.).

A “sensory and cognitive challenge state” as used herein is intended to encompass any state of the brain and central nervous system during the exposure to the sensory and cognitive challenge.

An “electronic system” as used herein is intended to encompass, without limitation, hardware, software, firmware, analog circuits, DC-coupled or AC-coupled circuits, digital circuits, FPGA, ASICS, visual displays, audio transducers, temperature transducers, olfactory and odor generators, or any combination of the above.

By “spectral bands” we mean without limitation the generally accepted definitions in the standard literature conventions such that the bands of the Power Spectral Densities (PSD) are often separated into the Delta band (f<4 Hz), the Theta band (4<f<7 Hz), the Alpha band (8<f<12 Hz), the Beta band (12<f<30 Hz), and the Gamma band (30<f<100 Hz). The exact boundaries of these bands are subject to some interpretation and are not considered hard and fast to all practitioners in the field.

By “calibrating” we mean the process of putting known inputs into the system and adjusting internal gain, offset or other adjustable parameters in order to bring the system to a quantitative state of reproducibility.

By “conducting quality control” we mean conducting assessments of the system with known input signals and verifying that the output of the system is as expected. Moreover, verifying the output to known input reference signals constitutes a form of quality control which assures that the system was in good working order either before or just after a block of data was collected on a human subject.

By “biomarker” we mean an objective measure of a biological or physiological function or process.

By “biomarker features or metrics” we mean a variable, biomarker, metric or feature which characterizes some aspect of the raw underlying time series data. These terms are equivalent for a biomarker as an objective measure and can be used interchangeably.

By “non-invasively” we mean lacking the need to penetrate the skin or tissue of a human subject.

By “electronics module” or “EM” or “reusable electronic module” or “REM” or “multi-functional biosensor” or “MFB” we mean an electronics module or device that can be used to record biological signals from the same subject or multiple subjects at different times. By the same terms, we also mean a disposable electronics module that can be used once and discarded, which may be part of the future as miniaturization becomes more commonplace and as the costs of production are reduced. The electronics module can have only one sensing function or a multitude (more than one), where the latter (more than one) is more common. All of these terms are equivalent and do not limit the scope of the invention.

By “diagnosis” we mean any one of the multiple intended uses of a diagnostic, including to classify subjects in categorical groups, to aid in the diagnosis when used with other additional information, to screen at a high level where no a priori reason exists, to be used as a prognostic marker, to be used as a disease or injury progression marker, to be used as a treatment response marker or even as a treatment monitoring endpoint. By “diagnosis” we also mean any of the ten intended uses that a biomarker can confer, which include but are not limited to: (1) as an aid in the diagnosis of a disease, disorder or condition, preferably early in a diagnostic algorithm; (2) as a prognostic marker to determine the likelihood or probability of a future event or brain condition later in time; (3) as a drug response marker to determine who might respond well to a candidate intervention or therapy before they start the therapy; (4) as a response to therapy marker for someone after they start an therapeutic intervention; (5) as a brain injury or disease progression marker to be quantitatively serially assessed over time to assess if things improve, deteriorate, stay the same or return back to normal; (6) as a non-invasive screening tool in healthy normal subjects to discover initial evidence of issues and problems; (7) as an at-home daily, weekly or other periodicity measurement to track longitudinal marker change within a subject; (8) as a drug compliance marker looking for a given benefit or signature that the therapy has been taken or conducted; (9) as a safety marker to show either the absence or presence of important changes in the safety profile of a human brain (for instance to document post-radiation encephalopathy or to evaluate the brain's response to chemotherapy (10) as a real-time monitoring marker (either full time or intermittent but regular with varying degrees of coverage).

By “multi-modal neuro-diagnostic scanning” we typically mean a diagnostic procedure that includes more than one modality of brain health assessment, often including two, three, or four modalities of biosignal data. In some instances, there can be five, six or seven different modalities of diagnostic information being collected. It should also be explicit that this term also includes the use of individual modalities of neuro-diagnostic scanning, such as EEG, MRI, PET, CT or SPECT in isolation.

By “a single dose” we typically mean only one dose, but it would be contemplated that dividing a single dose into N equivalently reduced Dose/N doses would be equivalent to the original dose and thus be equivalent to a “single” dose.

### Pharmaco-Diagnostic Multi-Modal Assessment

The systems and methods of the invention comprise utilization of a single diagnostic dose of a chemical agent such as a regulatory agency approved drug, biologic, vitamin, ingredient, or other chemical or biologic agent which can bind with molecular specificity or provide a well characterized molecular effect on a mammalian host (including humans). The systems and methods of the invention compare a first scan taken pre-dose to a second scan taken some period of time post-dose of the regulatory agency approved or acceptable chemical or biological moiety which confers molecular specificity. This dual scan process would then enable not only sensory, cognitive and physical assessment of the brain to various challenges, but it would also allow for a pre-dose versus a post-dose comparison of the molecularly specific biological challenge from the chemical agent or stimulus.

It should be noted that an embodiment of the invention also includes the use of this pre versus post scan sequence in conventional neuro imaging scanners, such as MRI, fMRI, CT, PET, SPECT, and MEG scanners. Although the conventional scanners do not have as many modes of information, it is clearly contemplated that this molecularly conferred diagnostic approach would be valuable in traditional neuro imaging modalities as well.

In practice, the conceptual approach is straightforward: conduct a baseline multi-modal assessment scan of an individual, have them take a single diagnostic dose of a molecularly specific agent, and then re-scan the human subject again for a second time in exactly the same fashion as the baseline. Typical delay times Tₚ between the scans would range between 15 to 60 minutes, but could be shorter or longer, after ingestion of the molecularly specific agent, but this time could be much longer if desirable based on the time constant characteristics of the therapies pharmacokinetics (PK) or pharmacodynamics (PD), depending on which properties are of interest. The pre-dose versus post-dose response will be compared across the multi-modal biosignal data streams and extracted features to identify univariate or multivariate signatures that can help classify, identify, prognosticate or otherwise help a doctor, nurse, ATC, EMT, or parent assess a patient, subject or themselves.

Moreover, the same generalizable approach can be used proactively in preparation for or in anticipation of the possibility of a putative “event” in the future, such as a concussion due to combat or sports or showing signs of memory loss from the aging process. The baseline scan would include a first pre-dose scan battery including physical, sensory, and cognitive challenge tasks. Then, a single dose of a regulatory agency approved or acceptable chemical, drug, biologic or therapy would be taken for diagnostic purposes. After an appropriate pause time Tₚ, which would depend on the physical PK properties as well as the pharmacodynamic (PD) properties of the chemical agent, an identical second post-dose scan would be conducted. A pre-dose versus post-dose comparison would provide a measure of response of that individual to the drug or molecular agent of choice. This molecularly specific response from the baseline pre-dose scan to Tp minutes post-dose scan can serve as a diagnostic biomarker of the neurotransmitter tone or molecular response of the human subject's brain.

For instance, if a single dose of an acetylcholinesterase inhibitor (e.g. Donepezil) is utilized, it would probe the cholinergic tone of the human subject's brain. Individuals with healthy brains would respond similarly while those with a cholinergic deficit or disorder would be revealed by their abnormal response to the acetylcholinesterase inhibitor. This comparison could be done looking at two visits from the same subject as a longitudinal within subject adjustment comparing a response years or weeks earlier in time from the same individual or alternatively as a cross-sectional normative comparison to an age, gender, handedness, etc., demographically created norm. Likewise, if a dopaminergic deficit was anticipated or hypothesized, one could select a dopamine specific agent to probe the dopamine system in the brain. In this way, a multi-modal assessment could gain molecular specificity when used in conjunction with a single dose of a molecularly specific agent.

It should also be clear that use of more than one molecular agent can be tested to assess the brain's response across multiple neurotransmitter systems. Thus, one could choose to probe the cholinergic, dopaminergic, serotonin, norepinephrine, glutamatergic, and GABA systems, a means to phenotype the neurotransmitter tone of the brain. As a non-limiting example, this can be accomplished either in a single cocktail of single dose agents (complicated by possible drug-drug interactions) or after multiple single dose assessments conducted after sufficient chemical agent wash-out to insure that each compound was probing uniquely for evidence of neurotransmitter system deficits or disorder with no cross-contamination from the previous compound or compounds. Because concussion or mild Traumatic Brain Injury (mTBI) in particular is a very heterogeneous brain injury, probing with several single diagnostic dose scans could help phenotype the injury, providing neurotransmitter level information.

Much later in time, say weeks later in a sports season or years later in one's senior years, when the human subject could have a putative concussive or memory loss event that requires a re-assessment by a physician or affiliated healthcare provider, it would be helpful to have scanned the subject first through a typical testing paradigm. Then, one could reassess the human subject again at a later date after a putative “event” to see if their multi-modal scans and responses to a molecular agent were significantly different in response to either the physical, sensory, cognitive challenges or tasks comprising the scan battery.

A schematic illustration of such a diagnostic scan session scheme is presented in the upper half of FIG. 1. With time in minutes along the x-axis of the upper timeline 2, a subject would undergo a pre-dose baseline multi-modal assessment scan of their brain 4 which could include any number of various sensory, cognitive, emotional, physical or other tasks, typically constructed to physiologically focus on the disease or diagnostic criteria known from previous studies including those identified in the published literature. After completing the baseline pre-dose multi-modal assessment scan 4, indicated to begin and end in time by the pair of vertical bars, a single diagnostic dose 6 of a molecularly specific agent would be administered according to the manufacturer's instructions. One would wait a time Tₚ, typically defined by the PK Tₘₐₓ or a PD effect time. At this pre-determined time point or time points, a second or subsequent but identical post-dose scan 10 would be conducted. A comparison would be done between the two scans looking typically at either the difference of a marker M equal to M₂−M₁ or the ratio M₁/M₂ of a given univariate marker or multi-variate composite signature.

If there is a distinct pharmacological response that is significantly different in a first state A of a brain compared to a second state B of a brain, then this difference can be used in any one of 6 or 8 of the different intended uses to help a healthcare practitioner (doctor, nurse, Certified Athletic Trainer, EMT, physical therapist, chiropractor, or parent as non-limiting examples) diagnose and manage a subject or patient. This general approach can be used in a multitude of diagnostic activities, depending on the nature of the disease/injury (and which neurological and neuropsychiatric systems are affected).

Standard biomarker intended uses include but are not limited to: (1) as an aid in the diagnosis of a disease, disorder or condition, preferably early in a diagnostic algorithm; (2) as a prognostic marker to determine the likelihood or probability of a future event or brain condition later in time; (3) as a drug response marker to determine who might respond well to a candidate intervention or therapy before they start the therapy; (4) as a response to therapy marker for someone after they start an therapeutic intervention; (5) as a brain injury or disease progression marker to be quantitatively serially assessed longitudinally over time to assess if things improve, deteriorate, stay the same or return back to normal; (6) as a non-invasive screening tool in healthy normal subjects to discover initial evidence of issues and problems; (7) as an at-home daily, weekly or other periodicity measurement to track longitudinal marker change within a subject; (8) as a drug compliance marker looking for a given benefit or signature that the therapy has been taken or conducted; (9) as a safety marker to show either the absence or presence of important changes in the safety profile of a human brain (for instance to document post-radiation encephalopathy or to evaluate the brain's response to chemotherapy); and (10) as a real-time monitoring marker (either full time or intermittent but regular with varying degrees of coverage). Note that continuously monitoring is equal to a 100% measurement duty cycle. Other intermittent patterns of monitoring could include: (i) 30 seconds of measurement every 5 minutes, thus 4.5 minute of no measurement along with=30/300=10% duty cycle; (ii) 30 seconds of measurement every 15 minutes would be equal to =30/900=3.33% duty cycle; and lastly as a non-limiting example, (iii) 30 seconds of measurement every 50 minutes=3000 seconds is a 30/3000=1% measurement duty cycle.

As is well-known in the art, there are six major neurotransmitter systems that compound interventions target, in which many common safe and effective drugs are already approved and could be used for pharmaco-diagnostic scanning. According to Dr. Gerard Sanacora, MD, PhD, Yale University School of Medicine, if one were to add the number of neurons that are using serotonin, norepinephrine, dopamine, and some acetylcholine, they usually account for ˜5% of the neurons in the brain, whereas glutamate and GABA make up ˜50% and ˜45%, respectively.” Thus, one can see that glutamate and GABA are the top two most important neurotransmitter systems, with Dopamine, Serotonin, Acetylcholine, and Norepinephrine rounding out the top six neurotransmitter systems.

In one particular embodiment of the present invention, the desire is to use immediate release drugs that exhibit short half-life, preferably those which have good PK properties such that their adsorption Tₘₐₓ is reasonable and practical from a dual scan perspective. This would facilitate looking at the difference or ratio pre-dose versus post-dose in a given subject in a time frame that is practical to implement in clinical care or home care setting. One skilled in the art will recognize that there are multiple diseases for which this approach can be successfully utilized.

### Parkinson's Disease

One particular embodiment of the present invention includes the use of carbodopa/levodopa, Pramipexole, Ropinirole, Bromocriptine or other similar dopaminergic acting regulatory agency approved drugs, biologics or active ingredients as a pharmacologic dose to probe the dopaminergic tone of the brain of a human patient or subject, in particular, for someone suspected of a dopaminergic brain disorder such as Parkinson's disease. Other compounds that that could be tested include, but are not limited to, biperiden, trihexyphenidyl, rasagiline, benztropine, entacapone, selegiline, rivastigmine, levodopa, rotigotine, bromocriptine, carbidopa/entacapone/levodopa, amantadine, selegiline, Apomorphine Hydrochloride, procyclidine, pergolide, and tolcapone. As described generally before, the analysis would compare pre-dose versus post-dose of a single dose at time points post single dose administration, such as T=30, 60, or 90 minutes post-dose versus pre-dose in the same subject to assess for pre-motor deficit evidence to aid in the diagnosis of a Parkinson's patient (ICD-9 code 332.0 or cross-walk equivalents). The pre-dose versus post-dose (i) difference and/or (ii) ratio “effect size” alone or in combination with other markers would form a multi-variate composite signature in an unknown individual would be compared to a first normative set of data from individuals classified in state A as well as potentially a second, third and/or fourth population of subjects with state B, state C and state D. In practice, the health care practitioner, most typically a neurologist but also a family physician or other licensed professional, would calculate the response of the human subject and compare the signature of the unknown subject to the comparator groups of state A, B, C, and D. Once a best match classification or regression to a number is found, this information can be provided in the form of a data and analysis report to licensed healthcare practitioners to aid in the diagnosis of the subject according to the various intended uses already discussed.

It is noted that normative comparator groups can be not only random in nature but the present invention also contemplates matching as demographically as possible the normative group to the unknown individual using simple co-variates such as gender, age, weight, height, smoking status, BMI, pulse rate, blood pressure or any other commonly available demographic or laboratory co-variate marker or variable. The process can alternatively incorporate very sophisticated selection algorithms that match analytically a given subject to the closest 300 subjects that look like them in a database of thousands to millions of individuals and then compare the unknown subject to their personalized closest 300 subjects to see if they are within the normal range for those individuals. The same approach can be used to pre-select the closest normative groups in each of states A, B, C, and D so that the comparison of unknown subject is to signatures created from the closest and most meaningfully controlled sub-sets of subjects in the super set of patients/subjects available. This provides then the opportunity to control for as many unknown variables as possible in the selection of the normative 300 subject population, as a non-limiting example.

### Concussion and Traumatic Brain Injury

Another important embodiment of the present invention includes the use of the selective serotonin reuptake inhibitors (SSRI) as a class of pharmaco-diagnostic agents, including citalopram, escitalopram, Fluvoxamine, paroxetine, fluoxetine, and sertraline, that could be used to probe the serotinergic tone of a human subject's brain as part of a multi-modal diagnostic response to a single dose of one of these agents, especially in the case of someone suspected of a concussion and/or traumatic brain injury. In addition, the present invention contemplates use of the tricyclic antidepressants including specifically Amitriptyline, Imipramine (Tofranil), Nortriptyline (Pamelor), Clomipramine (Anafranil), and Desipramine (Norpramin), as well as glutaminergic medications like methylphenidate or amphetamine, in addition to NMDA receptor antagonist Ketamine, lamotrigine (glumate modulator), Memantine, and riluzole. In particular, for severe TBI, a dopamine agonist, in the stimulant class, like Adderall would be diagnostically informative to assess the brain's response to a single diagnostic dose. One versed in the field will appreciate that there are other similar CNS active regulatory agency approved drugs, biologics or active ingredients that could be tested as a pharmacologic dose to probe the tone of the brain of a human subject. As described before, the analysis would compare pre versus post of a single dose at time points post single dose administration, such as T=30, 60, or 90 minutes post versus pre-dose in the same subject to assess for multi-modal biosignal evidence to aid in the diagnosis of a concussion or TBI (ICD-9 code 854.0 or cross-walk equivalents) as well as to aid in the management during the return to school (or return-to-learn), return to play, return to work or return to duty progression. It is also contemplated in the invention that in certain instances, multiple follow-up scans post-dose may be desired to understand more subtle dynamics and variations of a brain's response over time, rather than just in a single post-dose scan.

It is important to note that pre-dose versus post-dose (i) difference and/or (ii) ratio could serve to provide biomarker evidence to help clinical practice and improve outcomes. Improved health outcomes and reduced costs could be documented in the following fashion: (1) the number of days Nᵢ out of activity (school, sport, work, duty) could be counted for each case i with the expectation that those who were diagnosed and managed with the diagnostic device of the present invention would return to work, return to play, return to school (return to learn), and/or return to duty sooner than those who were not diagnosed or managed with the present device, as measured by the number of days out of activity; (2) one would document that the “return-to-activity” is safer when managed with the present invention than those cases which were managed without the diagnostic device as quantified by fewer cases or reduced frequency of negative health outcomes defined by deaths, paralysis, Post-Concussion Syndrome cases, Second Impact Syndrome cases, Depression cases, PTSD cases, or other adverse clinical outcome. Lastly, this invention could be used to identify fraud and “malingering” and thereby reduce time on benefits and increase return to activity as medically warranted.

### Cognition, Alzheimer's Disease, Dementia and Mild Cognitive Impairment

Another important embodiment of the present invention includes the use of the approved therapeutic agents for Alzheimer's disease in a pharmaco-diagnostic capacity. In particular, the class of acetylcholinesterase inhibitors including donepezil, tacrine, rivastigmine, and galantamine could be used to probe the cholinergic tone of a human subject's brain to test the multi-modal diagnostic response to a single dose of one of these agents. In addition, an NMDA-receptor antagonist like Memantine could alternatively be used to probe the tone in an alternate neurotransmitter system for important complementary or standalone diagnostic information.

One versed in the field will appreciate that there are other similar CNS active regulatory agency approved drugs, biologics or active ingredients that could be advantageously utilized as a pharmacologic diagnostic dose to probe the tone of the brain of a human subject. As described before, the analysis would compare pre-dose versus post-dose of a single dose at time points post single dose administration, such as T=30, 60, or 90 minutes post versus pre-dose in the same subject to assess for multi-modal biosignal evidence to aid in the diagnosis of Alzheimer's disease, Mild Cognitive Impairment, or other forms of dementia or cognitive disorders (ICD-9 codes 331.0, 331.83, 290.0 and those between 290.0-295 and 330.0-340, or cross-walk equivalent codes) as well as to aid in the management of the medical condition during the chronic phase which can last for years.

It is important to note that pre-dose versus post-dose (i) difference and/or (ii) ratio could serve to provide biomarker evidence to help clinical practice and improve outcomes. In this case, improved health outcomes and reduced costs could be documented in the following fashion: (1) safety could be established by preventing patients from continuing to drive beyond their appropriate time; (2) overall reduced cost to the healthcare system could be established by decreasing the resources necessary to watch after someone in an independent living environment with the subsequent and inevitable hospitalization and healthcare costs without benefit of the present invention compared to those who would utilize the present invention.

### Epilepsy and Seizure Detection

Another important embodiment of the present invention includes the use of epilepsy and seizure management agents to (i) aid in the diagnosis of seizures or epilepsy, (ii) identify those at risk for seizures or epilepsy and (iii) evaluate the patient for potential efficacy of an epilepsy and seizure management agent. In particular, agents such as Gabapentin, carbamazepine, fosphenytoin, divalproex sodium, acetazolamide, phenytoin, carbamazepine, felbamate, perampanel, levetiracetam, pregabalin, ezogabine, vigabatrin, carbamazepine, oxcarbazepine, ethosuximide, tiagabine, mephobarbital, ethotoin, phenytoin, topiramate, and trimethadione to identify non-limiting examples, can be used in single doses to probe the tone of the human subject's brain under assessment for clinical response evidence that they may be more susceptible to seizures and epilepsy or that one epilepsy and seizure management agent may prove more efficacious. One versed in the field will appreciate that there are other similar CNS active regulatory agency approved drugs, biologics or active ingredients that can be used as a pharmacologic dose to probe the tone of the brain of a human subject. As described before, the analysis would compare pre-dose versus post-dose of a single dose at time points post single dose administration, such as T=30, 60, or 90 minutes post-dose versus pre-dose in the same subject to assess for multi-modal biosignal evidence to aid in the diagnosis of epilepsy or seizures disorders (ICD-9 code 345.9, or cross-walk equivalents) as well as to aid in the clinical management of these patients once on the medication.

It is important to note that pre-dose versus post-dose (i) difference and/or (ii) ratio could serve to provide biomarker evidence to help clinical practice and improve outcomes. In this case, improved health outcomes and reduced costs could be documented in the following fashion: (1) earlier return to work through earlier return to driving; (2) decrease trial and error of antiepileptic medication prescription, as measured by reduced time (in days) to stable therapy or reduced number of medications trialed before achieving stable therapy; (3) reduce potential unnecessary use of antiepileptic medications; (4) decrease potential health hazard to patient and others on the road if the patient has an undiagnosed seizure while driving; and (5) limit the use of prolonged hospitalizations to identify seizure disorders.

### Autism and Other Neuro Development Disorders

Another important embodiment of the present invention includes the use of the NDMA and GABA therapeutic drugs to probe the tone of the developing human brain, in particular for evidence of Autism Spectrum Disorders (ASD) in infants and toddlers. In particular, agents which modulate metabotropic glutamate 5 receptor (mGluR 5) might serve well to help aid in the diagnosis and management of Autism Spectrum Disorder and other development neurological disorders.

One versed in the field will appreciate that there are other similar CNS active approved drugs, biologics or active ingredients that can be used as a pharmacologic dose to probe the tone of the brain of a developing human subject. As described before, the analysis would compare pre-dose versus post-dose of a single dose at time points post single dose administration, such as T=30, 60, or 90 minutes post-dose versus pre-dose in the same subject to assess for multi-modal biosignal evidence to aid in the diagnosis of neurological developmental disorders (ICD-9 code 315 or cross-walk equivalents) as well as to aid in the clinical management of these patients once on the medication.

It is important to note that pre-dose versus post-dose (i) difference and/or (ii) ratio could serve to provide biomarker evidence to help clinical practice and improve outcomes. In this case, improved health outcomes and reduced costs have been well documented in the following fashion: (1) when diagnosis is made early in life between 6 months and 24 months of age, cognitive behavioral therapy and other interventions has been shown to dramatically affect clinical outcome for the patient (Fein, D, et al, Optimal Outcome in individuals with a history of Autism. J Child and Adolescent Psychiatry 54:2 (2013); Dawson, G, et al, Randomized controlled trial of an intervention for toddlers with autism; the Early Start Denver Model. Pediatrics 125:e17 (2010)).

### Depression

Another important embodiment of the present invention includes the use of the approved SSRI therapeutic class of agents in a pharmaco-diagnostic capacity to aid in probing the serotonin tone, in particular, for neuropsychiatric conditions like depression. In particular, the class of SSRI's including citalopram, escitalopram, Fluvoxamine, paroxetine, fluoxetine, and sertraline could be used to probe the serotinergic tone of a human subject's brain to test the multi-modal diagnostic response to a single dose of one of these agents. In addition, an NMDA-receptor antagonist like Memantine could alternatively be used to probe the tone in an alternate neurotransmitter system for important complementary or standalone diagnostic information in the case of depression.

It is important to note that pre-dose versus post-dose (i) difference and/or (ii) ratio could serve to provide biomarker evidence to help clinical practice and improve outcomes. In this case, improved health outcomes and reduced costs could be documented in the following fashion: (1) safety could be established by preventing patients from continuing to drive beyond their appropriate time; (2) overall reduced cost to the healthcare system could be established by decreasing the resources necessary to watch after someone in an independent living environment with the subsequent and inevitable hospitalization and healthcare costs without benefit of the present invention compared to those who would utilize the present invention.

### GABAinergic Tone

Another important embodiment of the present invention includes the use of approved Gamma-Aminobutyric acid (GABA) inergic agents because GABA is the major inhibitory neurotransmitter in the mammalian Central Nervous System (CNS). The use of this therapeutic class of agents can be tested in a pharmaco-diagnostic capacity to aid in probing the GABAinergic tone, in particular for neuropsychiatric conditions like anxiety, schizophrenia, bi-polar disorder as well as pain and the cerebral cortex. In particular, the class of GABAinergic agents including GAD, GABA, Valerian, Gepirone, Buspirone, Sedatives, Zopiclone, Triazolam, Fengabine, Midazolam, Alprazolam, Adinazolam, Temazepam, Barbiturates, Methaqualone, Benzodiazepines, Neuroactive steroids, Clorazepam, lorazepam, and Diazepam (Valium), could be used to probe the GABAinergic tone of a human subject's brain to test the multi-modal diagnostic response to a single dose of one of these agents.

One versed in the field will appreciate that there are other similar CNS active approved drugs, biologics or active ingredients that could be advantageously utilized as a pharmacologic diagnostic dose to probe the tone of the brain of a human subject. As described before, the analysis would compare pre-dose versus post-dose of a single dose at time points post single dose administration, such as T=30, 60, or 90 minutes post-dose versus pre-dose in the same subject to assess for multi-modal biosignal evidence to aid in the diagnosis of neurologic and neuropsychiatric disorders as well as to aid in the management of their medical condition during a chronic phase which can last for years. It is important to note that pre-dose versus post-dose (i) difference and/or (ii) ratio could serve to provide biomarker evidence to help clinical practice and improve outcomes.

### Antipsychotic Medications and Assessment of Brain Tone

Another important embodiment of the present invention includes the use of approved antipsychotic agents in a pharmaco-diagnostic capacity to aid in probing the tone, in particular for neuropsychiatric conditions like anxiety, schizophrenia, schizoaffective disorders, OCD, Tourette's disease, tic disorders, bi-polar disorder and other mental health issues dealing with delusions, hallucinations, or disordered or disorganized thoughts. In particular, the class of antipsychotics agents including haloperidol, droperidol, chlorpromazine, fluphenazine, perphenazine, thioridazine, trifluoperazine, mesoridazine, triflupromazine, levomepromazine, promethazine, pimozide, cyamemazine, chlorprothixine, clopenthixol, flupenthixol, thiothixinem zuclopenthixol, clozipine, olanzapine, rispirdone, quetiapine, ziprasidone, amisulpride, asenapine, paliperidone, iloperidone, zotepine, sertindole, azipiprazole and lurasidone could be used to probe the tone of a human subject's brain to test the multi-modal diagnostic response to a single dose of one of these agents.

One versed in the field will appreciate that there are other similar CNS active regulatory agency approved drugs, biologics or active ingredients that could be advantageously utilized as a pharmacologic diagnostic dose to probe the tone of the brain of a human subject. As described before, the analysis would compare pre-dose versus post-dose of a single dose at time points post single dose administration, such as T=30, 60, or 90 minutes post versus pre dose in the same subject to assess for multi-modal biosignal evidence to aid in the diagnosis of neurologic and neuropsychiatric disorders as well as to aid in the management of their medical condition during a chronic phase which can last for years. It is important to note that pre-dose versus post-dose (i) difference and/or (ii) ratio could serve to provide biomarker evidence to help clinical practice and improve outcomes.

### Stimulants in the Assessment of Tone

Another important embodiment of the present invention includes the use of regulatory agency approved stimulant agents in a pharmaco-diagnostic capacity to aid in probing the tone of the brain. In particular, the stimulant class includes Adderall (amphetamine and dextroamphetamine), Dexedrine (amphetamine salts), and methylphenidate. It should be noted that non-stimulants could offer interesting diagnostic information from agents such as atomoxetine that could be used to probe the tone of a human subject's brain to test the multi-modal diagnostic response to a single dose of one of these agents.

One versed in the field will appreciate that there are other similar CNS active regulatory agency approved drugs, biologics or active ingredients that could be advantageously utilized as a pharmacologic diagnostic dose to probe the tone of the brain of a human subject. As described before, the analysis would compare pre-dose versus post-dose of a single dose at time points post single dose administration, such as T=30, 60, or 90 minutes post-dose versus pre-dose in the same subject to assess for multi-modal biosignal evidence to aid in the diagnosis of neurologic and neuropsychiatric disorders as well as to aid in the management of their medical condition during a chronic phase which can last for years. It is important to note that pre-dose versus post-dose (i) difference and/or (ii) ratio could serve to provide biomarker evidence to help clinical practice and improve outcomes.

### Norepinephrine Tone

Another important embodiment of the present invention includes the use of approved norepinephrine agents in a pharmaco-diagnostic capacity to aid in probing the Norepinephrinergic tone, in particular for neuropsychiatric conditions like depression, anxiety, schizophrenia, bi-polar disorder, ADHD and narcolepsy. In particular, the class of norepinephrinergic agents including tricyclic antidepressants such as Amitriptyline, Imipramine, Nortriptyline, Clomipramine, Imipramine, protriptyline and Desipramine for pain and headache, serotonin norepinephrine receptor inhibitors (SNRI) (serotonin>dopamine) such as duloxentine, venlafaxine, desvenlafaxine and milnacipran could be used to probe the Norepinephrine tone of a human subject's brain to test the multi-modal diagnostic response to a single dose of one of these agents.

One versed in the field will appreciate that there are other similar CNS active regulatory agency approved drugs, biologics or active ingredients that could be advantageously utilized as a pharmacologic diagnostic dose to probe the tone of the brain of a human subject. As described before, the analysis would compare pre versus post of a single dose at time points post single dose administration, such as T=30, 60, or 90 minutes post-dose versus pre-dose in the same subject to assess for multi-modal biosignal evidence to aid in the diagnosis of neurologic and neuropsychiatric disorders and conditions as well as to aid in the management of their medical condition during a chronic phase which can last for years. It is important to note that pre-dose versus post-dose (i) difference and/or (ii) ratio could serve to provide biomarker evidence to help clinical practice and improve outcomes.

### Sickle Cell Disease

Another important embodiment of the present invention includes its use at physician offices, field-derived locations or at home in order to proctor these brain health scans on a regular, longitudinal basis, either with or without use of a pharmaco-diagnostic agent, in order to monitor a Sickle Cell disease patient's brain for evidence of reduced or abnormal activity. For instance, weekly or twice a week monitoring of multi-modal brain health could provide objective evidence of changes taking place in a Sickle Cell patient such as cerebral ischemia, alteration in cerebral blood flow or neurocognitive decline. If certain brain derived biomarkers hit a trigger or cut-point derived from research studies which have been verified and validated in follow-up studies, then this at home scan could be used to cost-effectively monitor for when a therapy such as, but not limited to, blood transfusion is indicated, thus tailoring the therapy uniquely to the patient in a personalized medical fashion. Thus, the present invention also includes multi-modal biosignal evidence gathered in any one of an inpatient, outpatient or home/field setting.

One versed in the field will appreciate that there are other similar CNS active regulatory agency approved drugs, biologics or active ingredients that could be advantageously utilized as a pharmacologic diagnostic dose to probe the tone of the brain of a human subject. As described before, the analysis would compare pre-dose versus post-dose of a single dose at time points post single dose administration, such as T=30, 60, or 90 minutes post-dose versus pre-dose in the same subject to assess for multi-modal biosignal evidence to aid in the diagnosis of when a therapy such as, but not limited to, a blood transfusion is indicated in Sickle Cell disease (ICD-9 codes 282.60-282.7 or cross-walk equivalents).

It is important to note that pre-dose versus post-dose (i) difference and/or (ii) ratio could serve to provide biomarker evidence to help clinical practice and improve outcomes. In this case, improved health outcomes and reduced costs could be documented in the following fashion: (1) safety could be established by preventing patients from major adverse events like death, cerebral ischemia, neurocognitive decline, early disability and the inability to work. Moreover the present invention could serve to (2) improve patient satisfaction by reducing travel time and expense to visit an ER or physician's office when not managed properly.

### Postanoxic Encephalopathy

Another important embodiment of the present invention includes the use of multi-modal neuro diagnostic scanning, either with or without pharmaco-diagnostic CNS active agents, to provide prognostic biomarkers of a postanoxic encephalopathy subject who is in the emergency department or intensive care unit of a hospital, physician office or clinic. Prognostic markers could enable clinicians to predict patient outcomes and enable appropriate clinical decisions in light of the prognostic information. In particular, cardiologists, intensivists and neurologists may find the present invention useful in any patient who presents after an anoxic cerebral event such as, but not limited to, cardiac arrest, cardiac arrhythmia, near-drowning, respiratory failure or suicide attempt.

Modalities utilized presently at diagnosing, monitoring and prognosticating anoxic encephalopathy are limited and subject to interpretation. There is no diagnostically certain method yet of assessing the severity of an initial anoxic cerebral event. Nor is there any standard method for identifying those patients who will improve verses those who will not. Lastly, there is no diagnostically certain method to prognosticate postanoxic cerebral function in a long-term fashion.

One versed in the field will appreciate that there are other similar CNS active regulatory agency approved drugs, biologics or active ingredients that could be advantageously utilized as a pharmacologic diagnostic dose to probe the tone of the brain of a human subject. As described before, the analysis would compare pre-dose versus post-dose of a single dose at time points post single dose administration, such as T=30, 60, or 90 minutes post-dose versus pre-dose in the same subject to assess for multi-modal biosignal evidence to aid in the diagnosis and management of Postanoxic Encephalopathy (ICD-9 code 348.1 or cross-walk equivalents).

It is important to note that pre-dose versus post-dose (i) difference and/or (ii) ratio could serve to provide biomarker evidence to help clinical practice and improve outcomes. In this case, improved health outcomes and reduced costs could be documented in the following fashion: (1) identify patients who would benefit from more aggressive treatments; (2) identify patients that treatment would prove futile in regaining any meaningful neurological function; and (3) identify which pharmacological therapies would be most advantageous to the patient. Moreover the present invention could serve to (4) aid in the diagnosis, treatment and prognostication in patients who suffered an anoxic cerebral insult.

### Brain Related Insurance Benefits: Workers Compensation and Medicare Fraud

Another important embodiment of the present invention includes the use of multi-modal neuro-diagnostic scanning, either with or without pharmaco-diagnostic CNS active agents, to provide objective clinical evidence of impaired brain health or a lack thereof in relation to workers compensation insurance cases or Medicare/government insurance benefits related to brain injury and disorders, as well as other CNS related injuries like low back pain, amputation pain and neuropathic pain. Because many brain injuries are “invisible” as defined by the patient manifesting only subjective complaints without objective, corroborating diagnostic clinical evidence, it is very difficult today to tell who is legitimately hurt and in need of appropriate medical care and who is malingering and should get off insurance and return to work. Objective multi-modal diagnostic biomarker evidence from the present invention could enable clinicians and thus insurance companies to understand with objective clinical evidence who has suffered a brain-related or CNS associated injury and/or is still suffering from this injury.

In some instances, it will not be possible to have a baseline scan from which one can make a within-subject comparison. In those cases, one can use adaptive norms as described earlier from a focused demographically selected cohort selected on independent variables (age, gender, handedness, blood type, weight, height, zip code, etc.) to select the demographically closest 300 subjects based on an independent set of variables to the candidate as a comparator, then calculate their brain health norms on the neuro-focused variables of interest to compare to the unknown subjects results.

On the other hand, for those industries and occupations where brain or head injury is more commonplace, such as the following non-limiting occupations: (1) construction workers; (2) police and fire workers related to altercations and normal hazards on the job; as well as (3) military personnel, where one can compare before deployment brain scans to combat zone scans in order determine if there is true evidence of brain injury. In addition, when soldiers return home and enter the Veterans Administration or other similar governmentally administered health care, these earlier scans can provide objective clinical evidence of change in order to (i) support legitimate claims or (ii) refute illegitimate claims and thus prevent fraud and unwarranted expense. Also, the results of these scans can be correlated to data obtained during concussive injury in those soldiers wearing devices that record blast force and over-pressure.

In the case of Medicare, other governmental derived health insurance, or private payor health insurance, the screening scans can be used to legitimately determine the presence or absence of abnormal brain health in order to substantiate or reject enrollment claims for both short term and long term benefits. It should be emphasized that the “truth” is what the present invention seeks, such that those with brain injury should get appropriate care and benefits and those without brain injury should be prevented from receiving care and benefits fraudulently.

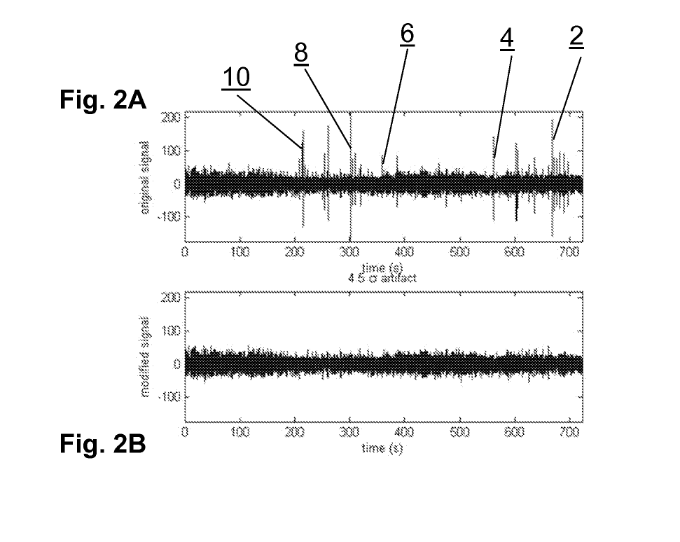

The aim would be to obtain a scan immediately after injury with as little scan interval (the time between the putative event and the scan) as possible. If a baseline scan was taken and stored prior to the putative event, direct comparison could provide an aid in the diagnosis of the concussive, brain-related or CNS-related injury, including low back pain and chronic pain. Alternatively, if one had several baselines scans profiling each of the dominant neurotransmitter systems (glutamate, GABA, dopamine, serotonin, acetylcholine, norepinephrine, etc.), then this phenotype or profile could be repeated post putative traumatic event and the neurotransmitter level information determined could objectively inform diagnosis, prognostication and case management. After the first scan post putative event, similar assessments can be done serially over time to aid in the management of the worker during the return to work progression (or in the case of the military, in return to duty decisions) or a determination of Maximum Medical Improvement (MMI, described below) as illustrated in FIG. 2.

FIG. 2 is a schematic diagram illustrating how the present invention could be used to manage return to work decisions, in a similar fashion to return to play decisions for athletes, return to learn decisions for students, or return to duty decisions for soldiers. This could be generally called “return-to-activity” decisions. As illustrated, a baseline diagnostic measurement Dx is taken at a point in time 20 prior to a putative accident or injury 22. A first diagnostic Dx assessment scan post-putative event is taken at a point in time 24 after the putative accident or injury 22. One or more subsequent Dx assessment scans may be taken at times 26 before a final Dx assessment scan it taken at time 28. After each scan, as appropriate, the scan results may be compared with previous scans to identify a change in the subject's brain, central nervous system and/or pain level as a result of the incident or injury that is outside a normal range of variation. Alternatively, the scan data may be used to estimate a probability that changes in the subject's brain, central nervous system and/or pain level as a result of said incident or injury are outside the normal range of variation. After the final assessment scan 28, the actual or estimated values may be used to determine whether the subject is ready to either return to work at 32 and/or whether the subject has reached MMI at 34. Similar assessments may be done for each of the examples set forth herein.

It should not go unnoticed that this pharmaco-diagnostic approach could also provide important neuro-diagnostic information to both plaintiffs and defense lawyers in order to substantiate evidence of injury (if the truth) or for insurance companies to substantiate evidence of no injury (if the truth) and prevent fraud. Moreover, lawyers themselves could utilize assessment scan information to decide if they want to take on a given case based on a simple and affordable multi-modal bio-sensor based scan that can be acquired by a certified and trained operator, such as a doctor, nurse, certified athletic trainer, physical therapist, pharmacist, or other careful and minimally trained data collector, typically an affiliated healthcare professional. This could also be helpful to insurance companies by helping the treating physician better determine through objective testing when a patient has reached “maximum medical improvement” and further medical treatments, testing and or office visits can no longer improve current care or outcome.

It should not go unnoticed that the multi-modal bio-sensor based assessment of the present invention can include any multiple combinations of a single, dual or few lead EEG device, ECG and heart rate variability measurements (since the brain and the heart are well linked between the two), respiration sensor: single or dual-band, actigraphy, audio, blood pressure, diary events, electroencephalogram, electromyogram, galvanic skin response (sometimes called galvanic skin conductance), pulse photo plethysmography & SpO2 (pulse oximetry), psychological testing by computer (e.g. CPT 96103), and neuropyschological testing by computer (e.g. CPT 96120). Others skilled in the art can ascertain there are additional wearable medical bio-sensors from which to generate additional multi-modal combinations.

The preferred data collection paradigm includes collection of a baseline clinical assessment data of the subject before an injury occurs. This could be stored but not analyzed to reduce cost and only analyzed in the event of a putative accident or injury. Then, post putative event/injury, another set of data can be collected in the same set or subsets of tests such that a direct within subject comparison is possible enabling a more informed and meaningful difference. Standard statistical analysis will be able to place a probability on the retest individual's difference from their baseline in order to help quantify the probability of a significant difference, which would offset any general inaccuracies in the bio-sensor measurements.

In the event it is not possible to obtain a baseline assessment before an injury occurs, an adaptive normative data set (so called “adaptive norm”) could be created and the subject in question compared to the adaptive norm established by the demographically matched peer group. For instance, if a 33-male, 6′2″, 185 pound construction worker with A-positive blood, married, non-smoker hurts their back on the job without a baseline assessment before the putative injury, one can gather electronically from the database of bio-sensor data records as many other 33 year old male, 6′2″, 185 pound, A-positive blood, married, non-smokers and assemble a norm from these individuals. One should loosen the criteria enough to enable at least an N=100 subjects in the adaptive normative data set, although fewer can be used in the beginning and preferably more like N=200, 300, 400, or 500 could be used later in time. Once the database is largely populated with millions of people, then thousands can be assembled or more refined adaptive norms can be used which include additional factors which further limit and filter the overall number of individuals assembled into the adaptive norm. Although this is not as exact and a within subject comparison, it is a better approach then assembling a norm from all males or all females as the case may be for any given subject.

### Insurance Benefit: Clinical Information to Manage Risk and Help Identify Fraud Generally

One particular embodiment of the present invention is about determining a risk factor, risk factors, adjusted probably or classification scheme about the objective truthfulness that employees and other insurance beneficiaries are self-reporting. In particular, this clinical information would be very helpful when it concerns subjective or invisible injuries, in particular soft tissue injuries, low back pain and brain related injuries (migraine, post-concussive headaches, post-concussive neurocognitive problems, and dizziness as non-limiting examples).

Because many of these complaints are subjective in nature, there are no validated diagnostic tests available to substantiate, quantify or corroborate these complaints. Thus, these complaints could be fraudulently claimed by the patient, embellished upon by the patient or exacerbated by co-morbidities such as underlying psychiatric pathology. Alternatively, these claims could be denied by the patient in order to return to prior activities even though they are at a greater risk of re-injury. This embodiment of the current invention would serve as a validated, diagnostic test to substantiate the injured person's subjective complaints, objectively follow the progression of these complaints and provide a valuable tool for the physician to make accurate, clinical determinations about the patient's symptoms.

### Insurance Benefit: Clinical Data to Aid in Injury Recovery Management and Return-to-Work

One particular embodiment of the present invention is used to manage the injury recovery of an individual subject and to enable better return-to-work decisions. In particular, a multi-modal bio-sensor based approach is used to generate clinical information about the status of an injury and use multiple measures throughout the recovery period to monitor objectively the progress a subject is making without having to only rely on the self-report by the individual themselves, which is often biased.

### Insurance Benefits: Clinical Data to Aid in the Determination of Maximum Medical Improvement

One particular embodiment of the present invention is about determining maximum medical improvement (MMI) and providing objective evidence to standardize maximum medical improvement (MMI) guidelines. Also, this embodiment could provide objective and quantifiable data to help a physician determine when medical therapy and/or treatment has reached a plateau in its efficacy. Because many invisible injuries, in particular soft tissue injuries, low back pain and brain related injuries (migraine, post-concussive headaches, post-concussive neurocognitive problems, and dizziness) are subjective in nature without quantifiable evidence to validate the patient's complaints, the determination of MMI by a physician is also subjective in nature and without any standardized format. Thus, physician's MMI determination has traditionally been physician-dependent, variable per practitioner and without a uniform, standardized consensus. With regard to this embodiment, the physician would be able to quantify and accurately follow the progression of these subjective complaints. By quantifying and accurately monitoring these complaints using this current embodiment, accurate guidelines could be created, based upon objective diagnostic information, which could standardize the criteria for the determination for MMI. Thus, the current, subjective physician determination of MMI would be replaced by guidelines based upon validated diagnostic criteria based on diagnostic information. The current embodiment would be utilized to create these guidelines that would help maximize physician's time with patients, reduce unnecessary testing, procedures and medication administration and minimize the variability in the physician's determination of MMI.

Insurance Benefit: Body and Physiology Stimulants and Activations to Employ while Scanning with a Multi-Modal Bio-Sensor Based System

Stimulation of the body can include any of the following non-limiting means of stimulating the nervous system: visual or sight, sound, drugs, thermal grill illusion (see (1) Thunberg T (1896) Förnimmelserna vid till samma stalle lokaliserad, samtidigt pågående köld-och värmeretning. Uppsala Läkfören Förh 2: 489-495, (2) Boettger M K et al, 2012. Increased cold and heat pain thresholds influence the thermal grill illusion in schizophrenia, European J of Pain, v17; 200-209 or (3) Lindstedt F et al 2012, Evidence for Thalamic Involvement in the Thermal Grill Illusion: An fMRI Study. PlosOne. olfaction, taste, occipital nerve stimulation (ONS), transcutaneous electrical nerve stimulation (TENS), transcranial doppler stimulation (TDS), hyperventilation, breathe holding, transcranial magnetic stimulation (TMS), transcranial electrical stimulation (TES), static balance tests such as the Balance Error Scoring System (BESS) or dynamic balance tests like the walk and turn 180 degree tests could be used. Various olfactory stimulants and even gastronomic stimulants could be used to probe the appropriate physiological response.

### Insurance Indication: Back, Neck or Other Pain

In the case of back, neck or soft tissue pain, direct manipulation of the tissue and probing for neuro-inflammation and in fact exacerbating the tissue to see an evoked response in the human subject's multi-modal biosensor data. This has been done clinically with expensive and cumbersome technologies like Positron Emission Tomography (PET) using Fluor-deoxyglucose-18 labeled tracers (FDG-PET). One non-limiting embodiment would be articulation of the back under load or strain (such as picking up a 20 lb weight off the floor) or the pressing of the head of a subject complaining of neck pain directly onto a vertical force plate and simultaneously recording (i) the force plate measurements (to quantify the stimulation) along with recording the various multi-modal response bio-sensors including but not limited to (ii) the EEG brainwaves, (iii) eye movements during the articulation including the transition from so-called pain-free to painful states, and (iv) a 3 axis accelerometer with or without a 3 axis gyrometer recording the motion of a subject, also from the pain-free to painful states, to determine if in fact there is objective clinical evidence of pain from measurements of the CNS. In this case, it would be very important to look for reproducibility in the onset of pain symptoms at a relatively consistent stimulus level; otherwise if the so-called onset of pain kept moving around a lot and showing enormous variance, then this would be an indication of potential fraud and dishonesty. If it was consistent in a quantitative fashion, it would identify a true, physiological response to pain and would be less consistent with fraud.

### Insurance Indication: Traumatic Brain Injury (TBI) or Concussion

In the case of TBI/concussion or head trauma, e.g. from a fall off a ladder at work, or by getting hit in the head by a moving machine, there are several established metrics that could be assessed. The subject could be put through a battery of tasks which physiologically focus on the cognitive elements, balance/vestibular elements, as well as the self-report elements. This may also include wearing an EEG headband and looking for evidence of a TBI in the transition to sleep where it has been reported that modulation in delta, theta, alpha and beta powers are irregular after TBI. When available for high risk professions like construction workers, police officers, firemen, football players, boxers, mixed martial arts fighters, war fighters, etc., it would be advantageous to obtain a baseline scan on a subject before injury and use this scan as a personal baseline for future comparison in the event of any kind of traumatic brain injury. Direct subject comparison (pre-injury to post-injury) would allow for the determination of how far in standard deviations they are from their baseline. An indication that someone was one (1) standard deviation from their baseline would be a 66% probability that this was in fact a different value than the baseline. A two (2) standard deviation shift would indicate a 95% probability that the second measurement was in fact different than the first with a false positive rate of less than 5%. This stringent bar is required in medical diagnostic cases but is not at all necessary in insurance risk management and return to work as this is only being used to monitor likelihood of truth and help identify fraudulent behavior. Stimulants that could be used include auditory stimulation and visual or photic stimulation which have been reported to exacerbate the condition.

In one embodiment of the TBI or concussion assessment, a so-called “tidal wave test”, which is a form of a modified Romberg test, could be employed where the subject is asked to close their eyes and shake their head vigorously back and forth left to right in the form of a head “no” response three to five times rapidly. A truly concussed subject will get dizzy by such a test and feel as if hit by a “tidal wave,” have difficulty with balance and vestibular activity. A normal person is not adversely affected. This could be further revealed by the EEG brainwave data, eye tracking measurements made immediately after the vigorous head movements, as well as by accelerometer based bio-sensor measurements taken during the actual vigorous head shaking and immediately preceding and following the head shaking. The comparison of three phases—(i) pre-stimulation, (ii) during stimulation and (iii) post-stimulation—allows one to examine each objective bio-sensor data stream for evidenced transitions that legitimately support or refute the subject's subjective medical claim.

In another embodiment, neurocognitive tests could be administered via computer without operator present and an EEG headset could record brainwave activity. Leading neurocognitive tests for concussion include but are not limited to Immediate Post-Concussion Assessment and Cognitive Testing (ImPACT, U. Pittsburgh Medical Center), Computerized Cognitive Assessment Tool (CCAT, Axon Sports, CogState), Concussion Resolution Index (CRI, Headminder Inc., NY, NY), Automated Neuropsychological Assessment Metrics system (ANAM, National Rehabilitation Hospital Assistive Technology and Neuroscience Center, Washington, D.C.), Concussion Vital Signs (CNS Vital Signs, marketed by Pearson).

Simultaneously, an eye tracking device could independently monitor the left and/or right eye positions in time, or alternatively calculate a mean position of the eyes together. An accelerometer in the EEG headset could measure accelerations during the course of time of interest, for instance at 100 samples/sec in all three directions (e.g. a 3-axis accelerometer). Various balance tests such as the static BESS test, or a dynamic “walk and 180 degree turn” test could assess the motion, brainwaves and eye movement of a subject. Pre-defined signatures already reported in the literature could be further developed into normative standards. Non-limiting examples include (a) lowered relative beta brainwave power in TBI/concussion (see (i) Slobounov, Cao, Sebastinelli, Clin Neurophysiol (2009) and (ii) McCrea M, Prichep L, Powell M R, Chabot R, Barr W B, (2010). Acute Effects & Recovery After Sports related concussion: A neurocognitive and quantitative brain electrical activity study. J Head Trauma Rehabil 25: 283-292) or (b) enhanced difficulty with saccadic eye movement in concussion (for instance the Pierce and King-Devick saccade tests, see Oride M K H et al, Am J. Optom & Physio Optics (1985). Reliability study of the Pierce and King-Devick Saccade Tests. v63: 419-424, or the developmental eye movement (DEM) test or an improvement on the DEM) or (c) decreased fixation time, or (d) increased motion variance could be used to identify and substantiate a legitimate TBI or concussion (a generalization of the Balance Error Scoring System, since errors intrinsically involve movement out of position (See Riemann B L, Guskiewicz K M, Shields E W, (1999). Relationship between clinical and force plate measures of postural stability. J Sports Rehabil. 8: 71-82 and Guskiewicz K M (2003). Assessment of postural stability following sport-related concussion. Current Sports Medicine Reports 2: 24-30.). These exact biomarkers could be used alone or in combination within a multi-variate predictive statistical model to help further support the claim of a TBI or concussion. Lacking each of these would be objective evidence not supportive of having a concussion and could enable an insurance company to conduct further tests to substantiate, or not, the brain injury or potential fraud taking place on the claim.

### Insurance Indication: Headache

In the case of a headache, one could attempt to stimulate the headache and objectively measure the evoked response to the stimulants. It has been reported that both auditory stimulation and visual stimulation can exacerbate or produce a headache in those prone to headaches. On the other hand, if a subject is making a fraudulent claim of a headache, then there would not be the objective evidence of a worsening condition or validated headache response to the auditory or photic stimulation. Cerebral PET scans have demonstrated localized cerebral activation of specific anatomical locations in the brain during the ictus of such headaches as migraines or clusters. These anatomical signatures produced by the PET scan clearly delineate specific brain regions which are activated during a headache. In this particular embodiment, a photic and/or auditory stimulation could be administered independently to a patient, while the patient is simultaneously being monitored by multi-modal bio-sensor system which includes an EEG headset, recording brainwave activity. In this case, it would be very important to look for reproducibility in the onset of pain symptoms at a relatively consistent stimulus level using both photic and/or auditory stimulation independently. If the results were consistent in a quantitative fashion and consistent with validation EEG signatures, it would legitimately support or otherwise refute the subject's subjective medical claim.

### Insurance Indication: Migraine

In the case of migraines, it is known to have a cardiovascular component so use of a pulse oximetry would be very helpful to characterize objectively an individual's response. Auditory stimulants, such as white or pink noise, binaural beats, isochronic tones and monaural beats could be used to attempt to induce the migraine and objectively measure the physiologic response. In the case of migraine, use of auditory and/or visual/photic stimulation could also be employed to bring onset of migraine and measure of physiologic change to then be tested for consistency, or not, with the self-report claims of the subject under test. Moreover, the thermal grill, in which metal fingers are interwoven and placed at either similar or dissimilar temperatures to create an artificial pain sensation can be used to look for heightened sensation to pain in those suffering compared to those who are not. A test like this is important to use because most folks are unfamiliar with the test and therefore it is more difficult to guess what the truly injured profile of behavior and response should be to mimic in an attempt to defraud the insurance company and employer. The best tests rely on the objective bio-sensor measurements which are much more difficult to intentionally adjust. Very few of us have the ability to control our brainwaves, nor to control our automated eye movements and voice. These inexpensively available bio-sensor streams should then form the basis of normal versus injured response.

### Insurance Indication: Vertigo and Dizziness

In the case of vertigo, one could use eye tracking technology to objectively confirm if vestibular issues are present within a given subject. This would be best arranged in both a static context (like holding difficult postures) as well as dynamically (like moving through obstacles or walking with “beer goggles” which simulate restricted and distorted vision). An accelerometer in the EEG headset could measure accelerations during motion, for instance at a rate of 20, 40, 60, 80, 100 or 120 samples/sec in all three directions (e.g. with a 3-axis accelerometer with or without a 3-axis gyrometer). Various balance tests such as the static Balanced Error Scoring System (BESS) test, “tidal wave test”, or a dynamic “walk and 180 degree turn” test could assess the motion, brainwaves and eye movement of a subject. Perhaps even just using a single 3-axis accelerometer to measure static balance in several postures with eyes closed could document the degree of stability, including enable an assessment of variance around a center-of-gravity or center-of-mass (COG/COM). Of course, fraudulent individuals will do their best to emulate someone with a vertigo or vestibular dysfunction, but quantitative analysis of the motion sampled should reveal subtle differences such as a stochastic nature to the variation (e.g. deviations from randomness due to their using their muscles to attempt to mimic a random pattern, such that statistical tests of randomness could be employed). Pre-defined signatures already reported in the literature could be further developed in normative standards as discussed earlier. One particular embodiment would utilize these one or more of these technical bio-sensor modalities to create univariate and multivariate signatures to validate and quantify the patient's dizziness and vertigo complaints in the above mentioned fashion.

### Insurance Indication: Neuro Cognitive Impairment

One of the most straightforward embodiments of the present invention would be the use of established as well as novel neurocognitive tests in the evaluation of a claim of neurocognitive impairment. Cognition is often broken down into six domains, including Verbal learning and memory, Nonverbal learning and memory, Executive function abilities, Language, Visuospatial abilities, and Sustained Attention. These tests would preferably be administered by a computer for standardization and consistency, also enabling more quantitative analysis not only of the correct responses (accuracy) but also the reaction times, how subjects make errors, and if there is an underlying pattern to the behavior or is it in fact truly impaired and injured. Established tests and batteries include those from Cantab, CogState, CNS NeuroVitals, the ANAM from the US military, etc. Individual tasks and instruments of interest could include but are not limited to the Mini-Mental State Exam (MMSE), the Montreal Cognitive Assessment, (MOCA), the CAMCOG, the Free and Cued Selective Reminding Task (FCSRT), Wechsler recall and delayed recall task, the California Verbal Learning Test—first or second edition (CVLT, CVLT-II), the Paced Auditory Serial Addition Test (PASAT) to name but a few. There is a helpful review of screeners by Cullen B et al, J Neurol Neurosurg Psychiatry (2007). A review of screening tests for cognitive impairment. 78: 790-799 as well as the NIH's Cognitive and Emotional Health Project: the Healthy Brain which lists Cognitive Measures (see http://trans.nih.gov/CEHP/hbpcog-list.htm). As previously described, the assessment of a subject would be compared to either their baseline assessment if it exists (so called baseline adjusted) or to a norm assembled from other subjects who are considered normal and not injured. In this way, one could estimate the probability that differences from baseline or norm were truly reflective of an injury or not. Moreover, return to work could be assessed by the same means looking for a return to baseline or a leveling off in performance over time indicative potentially of a maximum medical improvement.

### Insurance Indication: Tinnitis

In another embodiment of the present invention, one can apply the same sensory stimulations that are described above, but directed at subjects who are complaining of tinnitus, another subjective and fraud prone condition, especially for the military and the Veterans Affairs organizations. In this case, binaural beats, monaural beats, isochronic tones and other auditory stimulations can be applied to the tinnitus person's ears with high quality ear buds. If there is a registration between the auditory stimulus and characteristic brainwave changes associated with the pain (e.g. destruction of the alpha peak) in an eyes closed state, then this would provide objective evidence to support a claim of tinnitus. If there was not any change in response as a frequency sweep was conducted, then this would not support a case for tinnitus. If someone complained of tinnitus when the sweep was going on, the frequency of pain onset could be monitored and recorded. It could be assessed several times and one can look for consistency. If the pain is real, it likely onsets at the same frequency each time and shows a consistent pattern. If the pain onset frequency jumps around markedly, this clinical evidence would not likely support a tinnitus claim.

### Insurance Indication: Discovery of Co-Morbidities to Enable Further Risk Reduction

In another embodiment of the present invention, one can document co-variates input factors, like pre-existing disease conditions and co-mordiities to begin to develop more refined models and further reduce risk. In practice, one could begin to annotate a series of important co-morbidities either directly from patient input or alternatively from linkage to electronic medical records or electronic health records, whereby a list of ICD-9 diagnostic or CPT procedural codes could be generated for a given subject over the previous 1 year, 2 years, 3 years, 4 years or 5 years to build models that predict which ICD-9 codes and which CPT codes in a subject's past makes them more likely to have an injury on the job. With this sort of data mining model, additional care and prevention can be taken with those individuals that begin to fit a pattern based on previous claim history.

### Fatigue and Lethargy

Another important embodiment of the present invention includes the use of multi-modal neuro-diagnostic scanning, either with or without pharmaco-diagnostic CNS active agents, to provide objective clinical evidence of fatigue and lethargy. In particular, agents such as, but not limited to, modafinil, armodafinil and amantadine can be used in single doses to probe the tone of the human subject's brain under assessment for clinical response evidence that they may be more susceptible to fatigue and lethargy. Also, the previously mentioned agents used as single doses can be utilized to assess the brain response in a patient who suffers from fatigue and lethargy.

One versed in the field will appreciate that there are other similar CNS active regulatory agency approved drugs, biologics or active ingredients that can be used as a pharmacologic dose to probe the tone of the brain of a human subject. As described before, the analysis would compare pre-dose versus post-dose of a single dose at time points post single dose administration, such as T=30, 60, or 90 minutes post-dose versus pre-dose in the same subject to assess for multi-modal biosignal evidence to aid in the diagnosis of fatigue and lethargy (ICD-9 code 780.79 or cross-walk equivalents) as well as to aid in the clinical management of these patients once on the medication.

It is important to note that pre-dose versus post-dose (i) difference and/or (ii) ratio could serve to provide biomarker evidence to help clinical practice and improve outcomes. In this case, improved health outcomes and reduced costs could be documented in the following fashion: (1) minimize the negative effects of shift work on patient's health and job by identifying patients who would perform poorly under these circumstances or who has become impaired due to their shift work schedule; (2) prevent motor vehicle accidents by identifying when certain drivers should stay off the road such as truck drivers who had little sleep; (3) reduce air-related accidents by identifying which pilots are too impaired to fly a plane; and (4) reduce time off work by identifying which potential medications may cause or is causing patients excessive fatigue/tiredness and the inability to maintain properly their work schedule.

### Therapeutic Approach to TBI by Use of NMDA Receptor Antagonist Memandine or Equivalent

Another aspect of the present invention is the use of Memantine, an NMDA receptor antagonist to treat patients diagnosed with a concussion or mild Traumatic Brain Injury. The idea is that when a patient suffers a concussive event a cascade of biochemical events occur in the brain. The byproduct of this cascade is neurotoxic chemicals which promote, worsen and potentiate the deleterious effect the concussive injury has upon the brain. Because NMDA receptor antagonists such as, but not limited to, memantine block the NMDA channel in the central nervous system, it is hypothesized that it may counteract at least some of the deleterious effects that these neurotoxic chemical can have on the brain.

One versed in the field will appreciate that there are other similar CNS active regulatory agency approved drugs, biologics or active ingredients that could be advantageously utilized as a pharmacologic diagnostic dose to probe the tone of the brain of a human subject. As described before, the analysis would compare pre-dose versus post-dose of a single dose at time points post single dose or multiple dose administration, such as T=30, 60, or 90 minutes post-dose or on a regular interval such as days or weeks versus pre-dose in the same subject to assess for multi-modal biosignal evidence to aid in the diagnosis and management of TBI.

### Therapeutic Approach to Parkinson's by Use of Vitamin Therapy for Gait

Another aspect of the present invention is the use of vitamins to treat patients diagnosed with Parkinson's disease. The idea is that the antioxidant effects of vitamins could have a beneficial impact on the motor control and gait of a Parkinson's patient.

### EXAMPLES

While the above description contains many specifics, these specifics should not be construed as limitations on the scope of the invention, but merely as exemplifications of the disclosed embodiments. Those skilled in the art will envision many other possible variations that are within the scope of the invention. The following examples will be helpful to enable one skilled in the art to make, use, and practice the invention.

### Example 1

### Use of Sinemet to Aid in the Diagnosis of Parkinson's Disease

As described above, a physician could scan the brain of a patient using a multi-modal system and follow-up analytics of the type described above. A subject would be scanned pre-dose to gather a baseline assessment. Then they would be administered a single diagnostic dose of Sinemet and asked to wait in the waiting room for 30, 60, or 90 minutes or until the T_max of the drug, as known from the drug label and Pharmacokinetic studies. Then they would be scanned a second time in identical fashion. They could also be scanned a third, fourth and/or fifth time at earlier and later time points than T_max in order to derive a time series view of the effect of Sinemet. One would predict that a pre-motor deficit Parkinson's patient or an already diagnosed Parkinson's patient would exhibit an abnormal response to Sinemet because their dopamine tone is abnormal due to the neurodegenerative process associated with Parkinson's disease. Characteristic features of the before scan data in comparison to the after dose scan data, either as a difference between the two scans or the ratio between the two scans, could be used to provide objective biomarker information of an abnormal dopaminergic tone. This could aid in the enrollment process for clinical trials in Parkinson's disease as well as in clinical practice and management of the disease.

As an alternative to that described above, a physician could scan the brain of a patient using a conventional neuro-diagnostic system (e.g., EEG, fMRI, PET, SPECT, etc.) and follow the pre-dose vs. post-dose active pharmacologic agent protocol described above. A subject would be scanned pre-dose to gather a baseline assessment, for instance using a DAT PET ligand in a PET scanner or using a conventional 10-20 montage EEG. Then they would be administered a single diagnostic dose of Sinemet (or other active dopaminergic agent) and asked to wait in the waiting room for 30, 60, or 90 minutes or until the T_max of the drug, as known from the drug label and Pharmacokinetic studies. Then they would be PET scanned a second time in identical fashion with the DAT ligand or recorded in EC and EO states with the 10-20 montage EEG. They could also be scanned a third, fourth and/or fifth time at earlier and later time points than T_max in order to derive a time series view of the effect of Sinemet. One would predict that a pre-motor deficit Parkinson's patient or an already diagnosed Parkinson's patient would exhibit an abnormal or different response to Sinemet because their dopamine tone is abnormal due to the neurodegenerative process associated with Parkinson's disease. This would then be noted or documented as the characteristic features of the before-dose scan data would be different in comparison to the after-dose scan data. In the case of DATscan PET scans, either as a difference between the two scans or the ratio between the two scans, could be used to provide objective biomarker information of an abnormal dopaminergic tone. In the case of the conventional 10-20 montage EEG, this could done either on an electrode by electrode basis, a more global basis such as a coherence measures, or other comparator looking at the either the difference or ratio of the EEG data (pre versus post Sinemet dose). Other conventional neuroimaging modalities could be leveraged with the use of a single pharmaco-diagnostic active CNS agent. This could not only aid in the enrollment process for clinical trials in Parkinson's disease but also serve in clinical practice and management of the disease. This process would also apply to other neurological disease states whereby other novel tagged neurochemical ligands could be tested in a similar fashion to the DATscan ligand, described above, pre and post dosing of a particular CNS-active pharmacological agent.

### Example 2

### Use of Ice Cube or Other Cold Object to Aid in the Identification of Pain and Return-to-Work (RtW) Decisions

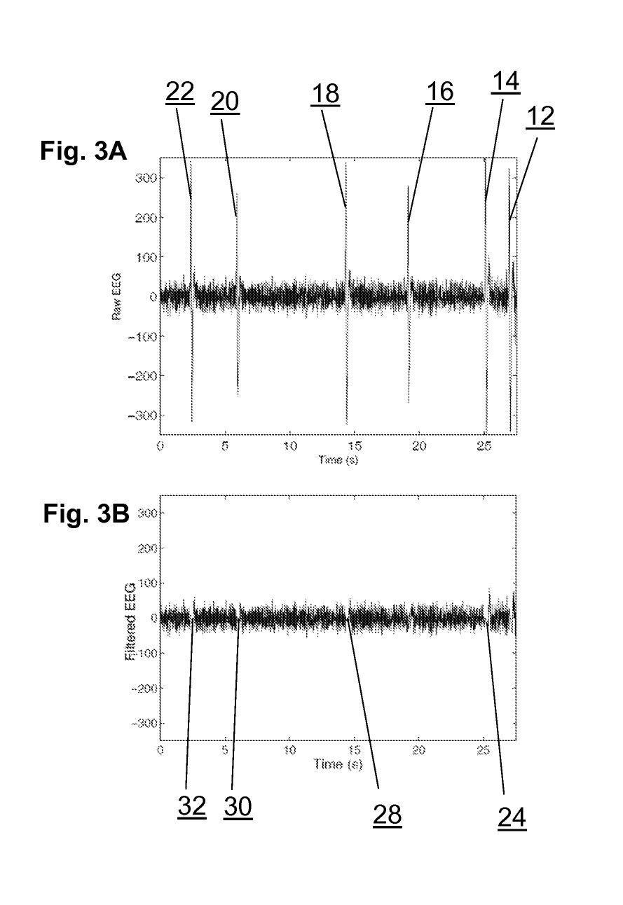

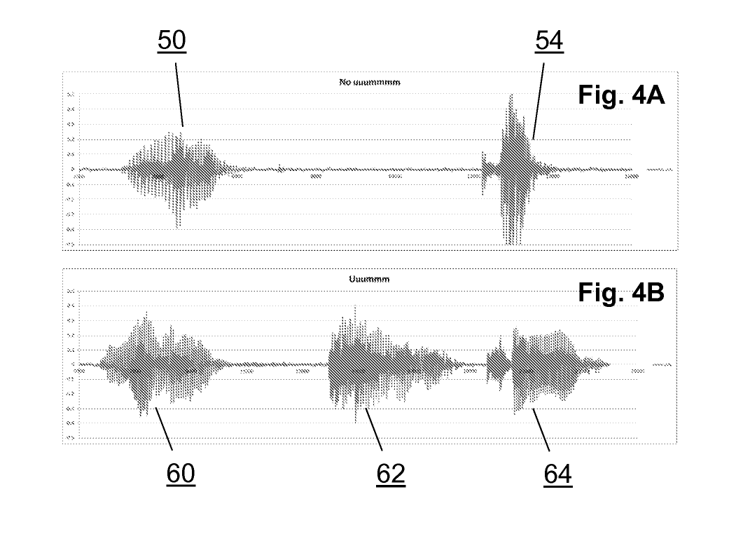

As described above, a physician could scan the brain of a patient using a multi-modal system and follow-up analytics of the type described above. A subject would be scanned pre-injury to gather a baseline assessment whenever possible. Then, if a claim of soft tissue related pain was made, one could use the methods of the present invention. As a non-limiting example, it is well known that in the resting eyes closed state, the alpha rhythm is prominent creating a peak often around 10 Hz. This was observed in a 45 old male subject as recorded by a single lead EEG headset with 128 samples/sec and 10-bit non-signed ADC (MindSet Pro from NeuroSky). As a surrogate for low back pain, or soft tissue damage and pain, the subject placed an ice cube in the left hand and held this for 60 seconds while the headset was recording. Although one can see a prominent alpha peak in the EC control power spectral density (See FIG. 3A, this alpha peak is significantly reduced when the pain from the ice cube is present, See FIG. 3B). FIG. 3 is a schematic diagram illustrating how the present invention could be used to manage risk and identify potential fraud in a work stream of claims within an insurance company for worker's compensation and other injury related claims by, for example, identifying the alpha peak in the scan data. This same paradigm could be used for the evaluation of back pain, asking a subject to sit comfortably with eyes closed, then lift a box with eyes closed, and then sit quietly with eyes closed. If the alpha peak is present in all conditions, then this would not be supportive of a pain claim. If it shifted while undergoing the EC lift condition, this would indicate some major shift. If it diminished, it would be consistent with the pain condition. For return-to-work decisions, this process could be monitored periodically, for instance every day, week or other frequency which the insurance company and/or physician should set. This would enable serial or longitudinal monitoring of the signal over time to determine when it has returned to baseline or alternatively, when it has flattened out and is now consistent over time.

### Example 3

### Use of a Thermal Grill to Aid in the Identification of Pain and Return to Work Decisions

As described above, a physician could scan the brain of a patient using a multi-modal system and follow-up analytics of the type described above. A subject would be scanned pre-injury to gather a baseline assessment whenever possible. Then, if a claim of soft tissue related pain was made, one could use the methods of the present invention. As a non-limiting example, it is well known that in the resting eyes closed state, the alpha rhythm is prominent creating a peak often around 10 Hz. This was observed in a 45 old male subject as recorded by a single lead EEG headset with 128 samples/sec and 10-bit non-signed ADC (MindSet Pro from NeuroSky). As a surrogate for low back pain, or soft tissue damage and pain, the subject could place their hand on a thermal grill, made of two independent pieces of metal with interlaced fingers held at various temperatures. It is known that pain circuits are sensitized when activated. Thus if a subject shows a heightened sensitivity to a thermal grill with mixed high and low temperatures, this would support a claim of pain related injury. As in the previous example, the prominence or lack thereof of the alpha peak is an excellent starting point. Other biomarkers can be identified that correspond to the painful state. This same paradigm could be used for the evaluation of back pain, asking a subject to sit comfortably with eyes closed on a thermal grill, then lift a box with eyes closed, and then sit quietly with eyes closed with their hand on a thermal grill. If the alpha peak is present in all conditions, then this would not be supportive of a pain claim. If it shifted while undergoing the EC lift condition or it became more sensitive to lower temperature difference between the two temperatures, this would support some shift. If it diminished, it would be consistent with the pain condition. For return-to-work decisions, this process could be monitored periodically, for instance every day, week or other frequency which the insurance company and/or physician should set. This would enable serial or longitudinal monitoring of the signal over time to determine when it has returned to baseline or alternatively, when it has flattened out and is now consistent over time.

### Example 4

### Use of Eye Tracking to Aid in the Identification of Vertigo and Return to Work Decisions

As described above, a physician could scan the brain of a patient using a multi-modal system and follow-up analytics of the type described above. A subject would be scanned pre-injury to gather a baseline assessment whenever possible. Then, if a claim of soft tissue related pain was made, one could use the methods of the present invention. As a non-limiting example, it is well known that vertigo, the eye movement of the subject is abnormal relative to healthy controls. As a surrogate for vertigo and dizziness, the subject could record their eye movement while standing still in place. Then, they could be asked to move slowly and record their eye movement. Then as a final return position, they could be asked to record eye movement at rest again. If in fact they have dizziness and/or vertigo, the saccadic movements of the eye, as evidence by the Pierce or King-Devick Saccade tests, Developmental Eye Movement test or an improvement on the DEM, should appear abnormal relative to their pre-injury or adaptive norm group. For return-to-work decisions, this process could be monitored periodically, for instance every day, week or other frequency which the insurance company and/or physician should set. This would enable serial or longitudinal monitoring of the signal over time to determine when it has returned to baseline or alternatively, when it has flattened out and is now consistent but not back to baseline over time.

### Example 5

### Prognostic Use to Predict which CNS Drugs Will be Most Efficacious

### Prophetic Example

Consider acquiring baseline scans in all patients undergoing psychiatric evaluation for a formal clinical diagnosis according to DSM-IV or DSM-V or improvements thereof. After the baseline scan, various medications would be prescribed to a patient on a case by case basis by their physician. Imagine noting which drugs were most efficacious for a given individual and classifying that patient by the drug that was eventually therapeutically effective. Imagine then running a predictive analytics exercise to evaluate which biomarkers from the baseline multi-modal assessment are predictive of patients going on to use a given outcome therapy effectively. Once this classifier is built, it could then be used on new patients after they undergo the baseline assessment to classify the new patient into to a given “most probable” outcome group associated with an effective therapy. Clinicians could then short cut therapy selection and rely on the baseline phenotype to discover which therapy is going to be probabilistically more effective for a given patient, shortening the time to stabilize therapy and improving the clinical outcome for the patient.

Those skilled in the art will appreciate that the invention may be applied to other applications and may be modified without departing from the scope of the invention. Accordingly, the scope of the invention is not intended to be limited to the exemplary embodiments described above, but only by the appended claims.

What is claimed:

1. A method of providing objective clinical evidence of impaired brain health or a lack thereof related to brain injury and brain disorders, neuro-degenerative disorders, central nervous system related injuries, insurance benefits, and/or pain, comprising:
providing a first multi-modal neuro-diagnostic scan of the subject to establish baseline objective multi-modal diagnostic biomarkers of the subject, said baseline biomarkers varying as a result of brain injury and brain disorders, neuro-degenerative disorders, central nervous system related injuries, and/or pain;
providing a second multi-modal neuro-diagnostic scan of the subject after an incident or injury or designated period of time; and
comparing the first and second scans to identify a change in the subject's brain, central nervous system and/or pain level as a result of said incident or injury or designated period of time that is outside a normal range of variation or to estimate a probability that changes in the subject's brain, central nervous system and/or pain level as a result of said incident or injury are outside the normal range of variation.

2. The method of claim 1, further comprising making a return to work, return to learn, return to driving, return to play, return to duty, return to activity or a continuation of insurance benefits decision in response to the comparison of said first and second scans.

3. The method of claim 1, further comprising providing the comparison of said first and second scans as objective evidence of a maximum medical improvement for the subject.

4. The method of claim 1, further comprising the step of providing diagnostic doses of one or more than one molecular pharmaco-diagnostic agent prior to at least one of said first and second scans, where said comparing step includes assessing changes or evaluating for stabilization/normalization in the subject's brain and/or central nervous system across one or multiple neurotransmitter systems.

5. The method of claim 4, wherein said molecular agent comprises at least one of:
selective serotonin reuptake inhibitors (SSRI), acetylcholinesterase inhibitors, NMDA-receptor antagonists, epilepsy and seizure management agents, NDMA and GABA therapeutic drugs in single diagnostic dose form, Gamma-Aminobutyric acid (GABA) ergic agents, antipsychotic agents, stimulant agents, and norepinephrine agents.

6. A method of providing objective clinical evidence of impaired brain health or a lack thereof related to brain injury and brain disorders, neuro-degenerative process, central nervous system related injuries, insurance benefits, and/or pain, comprising:
providing a normative data set of multi-modal neuro-diagnostic scan data of a plurality of subjects to establish objective multi-modal diagnostic biomarkers, said biomarkers varying as a result of brain injury and brain disorders, neuro-degenerative process, central nervous system related injuries, and/or pain;
providing a multi-modal neuro-diagnostic scan of the subject after an incident or injury or designated period of time; and
comparing the scan data to representative scan data from said normative data set to identify or to estimate the probability that the measured values from the scan data are outside a normal range of the corresponding values in the normative data set in the subject's brain, central nervous system and/or pain level as a result of said incident, injury or designated period of time.

7. The method of claim 6, further comprising making a return to work, return to learn, return to driving, return to play, return to duty, return to activity or a continuation of insurance benefits decision in response to the comparison of the scan data.

8. The method of claim 6, further comprising providing the scan data as objective evidence of a maximum medical improvement for the subject.

9. The method of claim 6, further comprising the step of providing diagnostic doses of one or more than one molecular pharmaco-diagnostic agents prior to said scan, where said comparing step includes assessing changes in the subject's brain and/or central nervous system across multiple neurotransmitter systems.

10. The method of claim 9, wherein said molecular agent comprises at least one of: selective serotonin reuptake inhibitors (SSRI), acetylcholinesterase inhibitors, NMDA-receptor antagonists, epilepsy and seizure management agents, NDMA and GABA therapeutic drugs in single diagnostic dose form, Gamma-Aminobutyric acid (GABA) ergic agents, antipsychotic agents, stimulant agents, and norepinephrine agents.

11. A method of providing objective clinical evidence of impaired brain health or a lack thereof related to brain injury and brain disorders, neuro-degenerative process, central nervous system related injuries, insurance benefits, and/or pain, comprising:
providing a first multi-modal neuro-diagnostic scan of the subject to establish baseline objective multi-modal diagnostic biomarkers of the subject, said baseline biomarkers varying as a result of brain injury and brain disorders, neuro-degenerative processes, central nervous system related injuries, and/or pain;
causing the subject to perform activities to activate an area of putative injury or pain;
providing a second multi-modal neuro-diagnostic scan of the subject after performance of said activities; and
comparing the first and second scans to identify a change in the subject's brain, central nervous system and/or pain level as a result of said activities that is outside a normal range of variation or to estimate a probability that changes in the subject's brain, central nervous system and/or pain level as a result of said activities are outside the normal range of variation.

12. The method of claim 11, further comprising making a return to work, return to learn, return to driving, return to play, return to duty, return to activity or a continuation of insurance benefits decision in response to the comparison of the scan data.

13. The method of claim 11, further comprising providing the comparison of said first and second scans as objective evidence of a maximum medical improvement for the subject.

14. The method of claim 11, further comprising the step of providing diagnostic doses of one or more than one molecular pharmaco-diagnostic agent prior to at least one of said first and second scans, where said comparing step includes assessing changes in the subject's brain and/or central nervous system across one or multiple neurotransmitter systems.

15. The method of claim 14, wherein said molecular agent comprises at least one of: selective serotonin reuptake inhibitors (SSRI), acetylcholinesterase inhibitors, NMDA-receptor antagonists, epilepsy and seizure management agents, NDMA and GABA therapeutic drugs in single diagnostic dose form, Gamma-Aminobutyric acid (GABA) ergic agents, antipsychotic agents, stimulant agents, and norepinephrine agents.

16. A method of providing objective clinical evidence of impaired brain health or a lack thereof related to brain injury and brain disorders, neuro-degenerative process, central nervous system related injuries, insurance benefits, and/or pain, comprising:
providing a first multi-modal neuro-diagnostic scan of the subject to establish baseline objective multi-modal diagnostic biomarkers of the subject, said baseline biomarkers varying as a result of brain injury and brain disorders, neuro-degenerative processes, central nervous system related injuries, and/or pain;
administering a diagnostic dose of an active dopaminergic agent to the subject;
providing a second multi-modal neuro-diagnostic scan of the subject after a period of time sufficient for the dopaminergic agent to become active; and
comparing the first and second scans to identify a change in the subject's brain, central nervous system and/or pain level as a result of said dopaminergic agent that is outside a normal range of variation or to estimate a probability that changes in the subject's brain, central nervous system and/or pain level as a result of said dopaminergic agent are outside the normal range of variation.

17. The method of claim 16, further comprising providing a plurality of multi-modal neuro-diagnostic scans of the subject over time to derive a time series view of the effect of said dopaminergic agent.

18. A method of providing objective clinical evidence of impaired brain health or a lack thereof related to brain injury and brain disorders, neuro-degenerative process, central nervous system related injuries, insurance benefits, and/or pain, comprising:
providing a first multi-modal neuro-diagnostic scan of the subject to establish baseline objective multi-modal diagnostic biomarkers of the subject, said baseline biomarkers varying as a result of brain injury and brain disorders, neuro-degenerative processes, central nervous system related injuries, and/or pain;
administering a diagnostic dose of a medication to the subject that is being tested for therapeutic effectiveness for the subject;
providing a second multi-modal neuro-diagnostic scan of the subject after a period of time sufficient for the medication to become active; and
comparing the first and second scans to identify a change in the subject's brain, central nervous system and/or pain level as a result of said medication that is outside a normal range of variation or to estimate a probability that changes in the subject's brain, central nervous system and/or pain level as a result of said medication are outside the normal range of variation to establish whether said medication is therapeutically effective for the subject.

19. The method of claim 18, further comprising repeating the providing, administering, providing and comparing steps for a plurality of medications to establish which medications are therapeutically effective for the subject.

20. The method of claim 19, further comprising running a predictive analytics exercise against data collected for the therapeutically effective medications to evaluate which diagnostic markers from the baseline multi-modal neuro-diagnostic scans are predictive of therapeutic effectiveness of the medications for use by another subject.
